(19) United States
(12) Patent Application Publication
(10) Pub. No.: US 2018/0174678 A1
(43) Pub. Date: Jun. 21, 2018

(54) IMAGE ANALYSIS MANAGEMENT METHOD AND SERVER FOR MEDICAL TESTS

(71) Applicant: THE CATHOLIC UNIVERSITY OF KOREA INDUSTRY-ACADEMIC COOPERATION FOUNDATION, Seoul (KR)

(72) Inventors: Kyung Ja HAN, Gwacheon-si, Gyeonggi-do (KR); Won Bae LEE, Gwacheon-si, Gyeonggi-do (KR)

(21) Appl. No.: 15/736,888

(22) PCT Filed: Jun. 16, 2016
(86) PCT No.: PCT/KR2016/006411

§ 371 Date: Dec. 15, 2017

(30) Foreign Application Priority Data
Jun. 19, 2015 (KR) 10-2015-0087482

(51) Int. Cl. G16H 30/40; G16H 50/20; G06N 99/00; G06T 7/00

(52) U.S. Cl. CPC G16H 30/40; G16H 50/20; G06N 99/005; G06T 2207/20081; G06T 2207/10056; G06T 2207/30101; G06T 7/0012

(57) ABSTRACT

Provided is a technology for medical tests wherein microcells are tested, which allows remote samples to be tested in one integrated testing place automatically and manually and enables learning based on test results therefor, thereby increasing the accuracy of automatic medical testing and maximally reducing limitations on the manpower, time, and place for manual and automatic analyses. The image analysis management method for medical tests according to one embodiment of the present invention comprises steps in which an image analysis management server for medical tests: receives, from a terminal provided in a testing place, an image captured with a microscope of a sample extracted for a preset medical test; receives, from an automatic analysis device or the terminal of a manually analyzing manager, test result data on the analysis of the received image using any one analysis method between analysis by the automatic analysis device and manual analysis; and transmits the received test result data to the terminal provided in the testing place.

### TECHNICAL FIELD

The present inventive concept relates to a method of managing a system for analyzing images for medical tests, for example, white blood cell analysis, gynecologic cancer diagnosis test, chromosome analysis, etc., in which high resolution images are used, and a system therefor, and more particularly, to a technology capable of integrally managing microscope images processed in various places and by using various devices and automatically reproducing a test result.

### BACKGROUND ART

In tests in medical fields such as sample analysis of white blood cells existing in human bodies, pathology tests such as gynecologic cancer diagnosis test, etc., and chromosome analysis, a medical doctor, a pathology doctor, and a clinical pathologist generally executes the tests by using a microscope based on sizes of white blood cells, cancer cells, chromosomes, etc.

However, there are limitations in human resources, places, etc. to perform the analysis through microscope observation, and thus, problems such as poor test efficiency and high costs have been shown. Accordingly, a device for automatically analyzing sample images captured by a microscope by using software to analyze samples of white blood cells, for example, Hemacam, has been recently developed.

However, such a device has a very low accuracy in identifying whether a sample is abnormal, in classifying samples, and in recognizing samples, and thus, a sample that may not be read by the device has to be manually analyzed by a doctor or a clinical pathologist. Also, the device is very expensive and the analysis may be performed only on an installation place, and thus, there are poor effects in improving the above limitations in the human resources, places, etc.

### DETAILED DESCRIPTION OF THE INVENTIVE CONCEPT

### Technical Problem

The present inventive concept provides a technology capable of automatically and manually testing a remote sample in an integrated testing place in a medical test for testing microcells, enabling learning of test results to improve accuracy in an automatic medical test, and at the same time, reducing limitations in human resources, time, and places of the manual analysis and the automatic analysis as much as possible.

### Technical Solution

According to an aspect of the present inventive concept, there is provided an image analysis management method for medical tests, the image analysis management method including: receiving, by an image analysis management server for medical tests, an image of a sample photographed by a microscope, the sample being taken for a preset medical test, from a terminal provided at a testing place; receiving test result data obtained by analyzing a received image through one of an analysis of an automatic analysis device and a manual analysis, from the automatic analysis device connected to the server or a terminal of a manager performing the manual analysis; and transmitting received test result data to the terminal provided at the testing place.

According to an aspect of the present inventive concept, there is provided an image analysis management server for medical tests, the image analysis management server including: an image receiver configured to receive an image of a sample photographed by a microscope, the sample being taken for a preset medical test, from a terminal provided at a testing place; a test result receiver configured to receive test result data that is obtained by analyzing a received image through one of an analysis by an automatic analysis device and a manual analysis, from the automatic analysis device or a terminal of a manager performing the manual analysis; and a test result transmitter configured to transmit received test result data to the terminal provided at the testing place.

### Advantageous Effects of the Inventive Concept

According to the present inventive concept, images captured by a microscope, etc. only are transferred to a remote integrated management server, result data is generated by analyzing the images through automatic analysis of an automatic analysis device in the management server or manual analysis performed by a doctor or a clinical pathologist, and after that, the result data is transmitted to a testee and a terminal located at a place requesting the test.

Accordingly, images captured in various places may be integrally analyzed, and thus, limitations in human resources, time, and places for analyzing microcells may be reduced. Also, the automatic analysis device automatically learns analysis results of a doctor or a clinical pathologist to improve an analysis performance thereof, and thus, efficiency of the automatic analysis device may be improved, and efficiency in tests requiring high accuracy, for example, white blood cell test, gynecologic cancer diagnosis test, chromosome test, etc., may be improved.

### DESCRIPTION OF THE DRAWINGS

FIGS. 1 to 3 are flowcharts of an image analysis management method for medical tests, according to an embodiment of the present inventive concept.

FIG. 4 is a block diagram of an image analysis management server for medical tests, according to an embodiment of the present inventive concept.

### BEST MODE

Hereinafter, image analysis management method and server for medical tests according to an embodiment of the present inventive concept will be described with reference to accompanying drawings.

For accurate understanding of the present inventive concept, a description of a publicly known technology for a characteristic of the present inventive concept will be omitted in the description below. The embodiments below are detailed descriptions for helping the understanding of the present inventive concept, and do not limit the scope of the present inventive concept as a matter of course. Accordingly, an equivalent inventive concept performing the same function as that of the present inventive concept also belongs to the scope of the present inventive concept.

In the description below, the same reference numeral denotes the same element, and unnecessary and overlapping descriptions and a description of a publicly known technology will be omitted.

In an embodiment of the present inventive concept, “communication”, a “communication network”, and a “network” may be used with the same meaning. Those three terms denote wired/wireless short-range and broadband data transceiving networks, which may transceive a file between a user terminal, terminals of other users, and a download server.

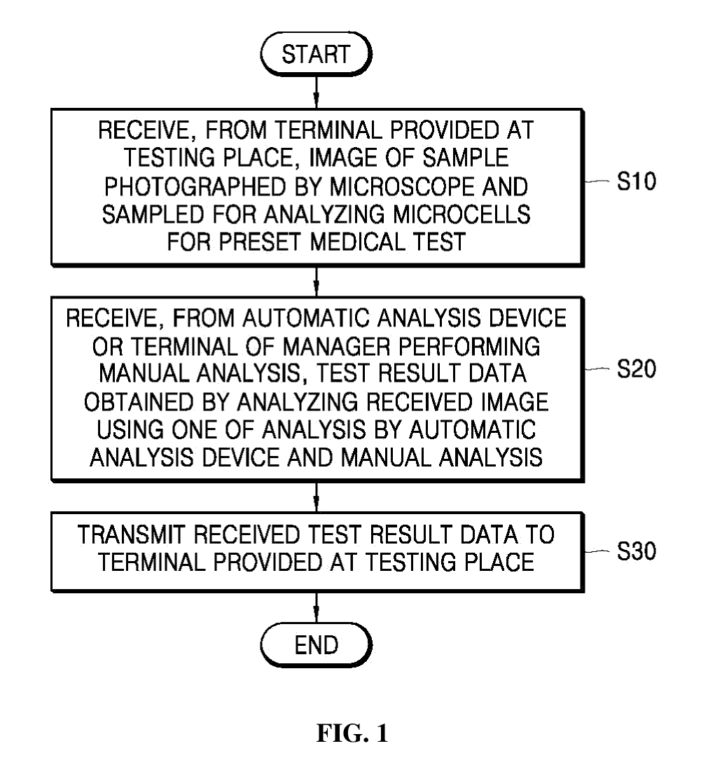

FIGS. 1 to 3 are flowcharts of an image analysis management method for medical tests, according to an embodiment of the present inventive concept.

The image analysis management method for medical tests according to an embodiment of the present inventive concept may be executed by an image analysis management server for medical tests (hereinafter, referred to as a server) according to an embodiment of the present inventive concept that will be described below. The server may include one server computer, or may include two or more computers according to functions thereof and amount and kind of data to be processed. Also, the server is connected to various medical devices and test devices via wired/wireless networks to transmit/receive data that is necessary for performing functions of the present inventive concept.

Also, in the present inventive concept, the medical tests denote tests for magnifying a sample at high magnification by using a high-resolution image capturing unit such as a microscope, etc., to determine whether cells, chromosomes, etc. included in the sample are abnormal or to classify the cells, chromosomes, etc. For example, a test for classifying white blood cells and analyzing abnormality of the white blood cells, a gynecologic cancer diagnosis test, other chromosome analysis tests, etc. in a field of pathology may be included in the medical tests of the present inventive concept.

That is, as mentioned above, the medical tests of the present inventive concept denote tests that a doctor or a clinical pathologist produces an analysis result by using a device such as a microscope, etc., by which a sample may be magnified at high magnification and observed, or an automatic analysis device using an algorithm of criteria of determination made by a doctor or a clinical pathologist automatically analyzes a high-magnification image of the sample and produces a test result through the test.

Based on the above grounds, referring to FIG. 1, the image analysis management method for medical tests according to the embodiment of the present inventive concept includes an operation, in which the server receives an image of a sample for a preset medical test, wherein the image is captured by a microscope, from a terminal provided in a testing place remotely separated from the server (S10).

The testing place is a remote place from the server, and a place where a microscope and a terminal that may receive an image from the microscope and transmit the received image to the server are provided. For example, a hospital that is located at a remote place from a hospital where the server is located and lacks of testing performance may correspond to a testing place.

A terminal provided at the testing place generates an image of a sample photographed by a microscope and enlarged at high magnification as a data format or receives a generated image, and transmits the generated image to the server. For example, the terminal may be a terminal of a doctor or a medical staff provided in the testing place. As will be described later, the terminal has to receive test result data.

After operation S10, the server receives test result data from an automatic analysis device connected to the server or a terminal of a manager performing manual analysis, wherein the test result data is obtained by analyzing the received image via one of the analysis of the automatic analysis device and the manual analysis (S20).

The automatic analysis device denotes all kinds of devices producing result data of medical tests by automatically analyzing a high-magnification image obtained by photographing a sample in a field included in the medical test of the present inventive concept, wherein the devices include a device automatically performing white blood cell classification and abnormality analysis as aforementioned above.

In addition, the terminal of the manager includes all kinds of terminals to which the manager performing manual review directly inputs the test results, for example, a terminal of a clinical pathologist connected to the server and performing the test.

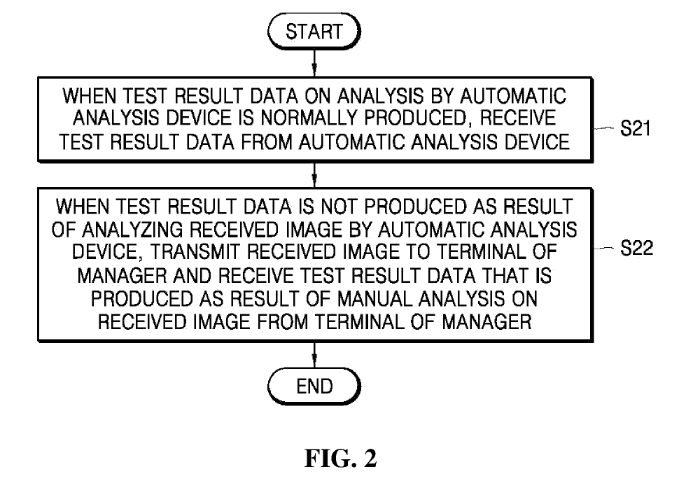

In the present inventive concept, the test result data produced by the automatic analysis device and the test result data input through the terminal of the manager may be input to the server through a flow shown in FIG. 2.

Referring to FIG. 2, if the test result data analyzed by the automatic analysis device is normally produced, the server receives the test result data from the automatic analysis device (S21).

A case where the test result data is normally produced is a case where the test result is clearly produced by the automatic analysis device, and may include a process of producing the test result data through the automatic analysis device and a process of approving the test result data by the terminal of the manager.

As described above, although the test result data may be produced by the automatic analysis device that is currently used, accuracy of the result is not guaranteed. Therefore, operation S21 may include a step of approving the test result data by the terminal of the manager as described above to improve the accuracy.

In addition, during operation S21, in a case where it is determined that the test result data that is obtained by analyzing the received image in the automatic analysis device is not normally produced, the received image is transferred to the terminal of the manager, and the server receives test result data produced as a result of manual analysis (manual review) on the received image from the terminal of the manager (S22).

In detail, in operation S21, the test result data produced by the automatic analysis device may be transmitted to the terminal of the manager. Here, in order to improve the accuracy of the test result, the received image may be also transferred to the terminal of the manager in operation S21. In this case, the process of transmitting the received image to the terminal of the manager may be omitted in operation S22.

Meanwhile, the case where the test result data is not normally produced may also include a case in which an approval is not input from the terminal of the manager although the test result data is produced, in addition to the case in which the test result data is not produced from the automatic analysis device.

That is, the automatic analysis device produces the test result through a preset algorithm. Here, it will be determined that the normal test result of the automatic analysis device has been produced only when the approval input of the manager is received, and if not, only the test result data transmitted from the terminal of the manager is used.

Referring back to FIG. 1, when receiving the test result data from the automatic analysis device or the terminal of the manager, the server transmits the received test result data to the terminal provided in the testing place (S30), and thus, the test result data obtained through professional and precise tests may be used in the remote testing place.

According to the embodiment illustrated in FIGS. 1 and 2, the testing place does not need the automatic analysis device or professional pathologists. That is, once the automatic analysis device and human resources are provided in an integrated test and management center in which the server is installed, the medical tests requiring professional analyzing performance may be performed anywhere in the world via a simple configuration of a microscope and a terminal.

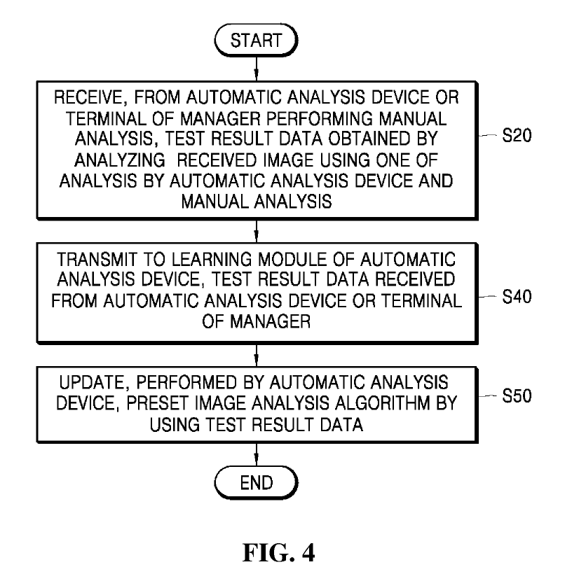

In addition, in order to maximize the performance of the automatic analysis device and reduce waste of the human resources, the server according to the present inventive concept may use a learning function of the automatic analysis device. Functional flows therefor are shown in FIG. 3.

Referring to FIG. 3, the server performs operation S20 of receiving the test result data mentioned in the above descriptions about FIGS. 1 and 2, and then, the server transmits the test result data received from the automatic analysis device or the terminal of the manager to a learning module of the automatic analysis device (S40).

In the embodiment of FIG. 3 of the present inventive concept, the automatic analysis device analyzes the received image through a preset image analysis algorithm provided therein in advance according to a kind of the medical test to produce the test result data, as described above. Also, the automatic analysis device may include the learning module that may update the above image analysis algorithm that is preset.

In detail, the learning module may store test result data that has been produced earlier as a database, that is, big data. The learning module generates the image analysis algorithm by using the test result data stored in the database and an initially input algorithm, and uses the image analysis algorithm in the test.

When the learning module of the automatic analysis device receives the test result data in operation S40, the automatic analysis device may update the preset image analysis algorithm by using the test result data (S50).

As described above, since the automatic analysis device performs the test by using the preset algorithm, it is difficult to guarantee accuracy of the test. Therefore, according to the present inventive concept, the test result data having guaranteed accuracy, e.g., manual analysis result received from the terminal of the manager is fed-back to the automatic analysis device, and the algorithm is updated via the learning module by using the manual test result in order to maximize the performance of the automatic analysis device. Accordingly, consumption of the human resources and time may be reduced and the efficiency in the test may be improved.

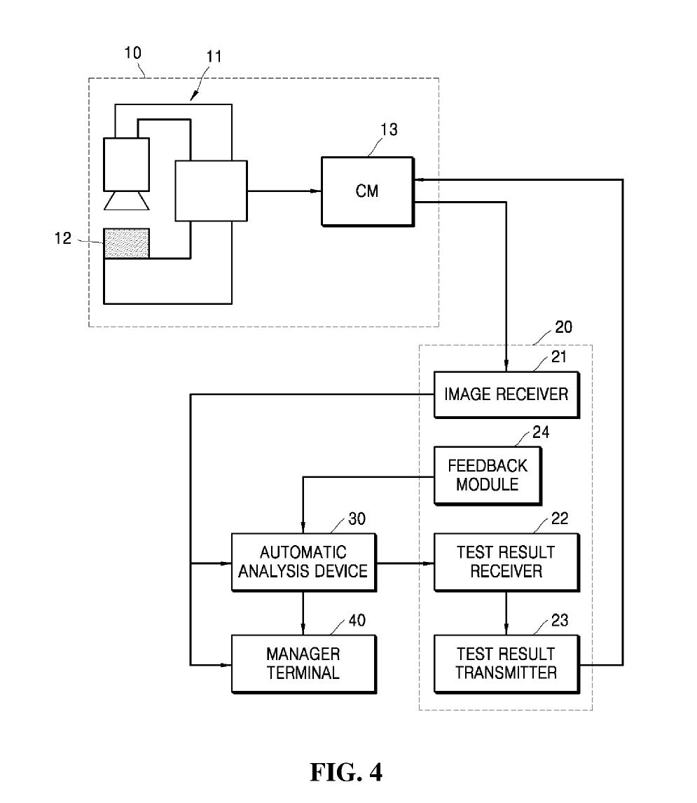

FIG. 4 is a block diagram of an image analysis management server for medical tests, according to an embodiment of the present inventive concept. Hereinafter, descriptions overlapping with the above descriptions with reference to FIGS. 1 to 3 will be omitted.

Referring to FIG. 4, an image analysis management server for medical tests 20 (hereinafter, referred to as a server 20) according to an embodiment of the present inventive concept may include an image receiver 21, a test result receiver 22, and a test result transmitter 23, and may further include a feedback module 24 for performing the same functions as those of FIG. 3.

The image receiver 21 performs a function of operation S10 described above with reference to FIG. 1, that is, receives a high-magnification image of a sample 12 photographed by a microscope 11 from a terminal 13 provided at a testing place 10.

The test result receiver 22 receives test result data that is the analyzing result of a received image performed by an automatic analysis device 30 and a terminal 40 of a manager who performs manual analysis. Here, functions illustrated with reference to FIG. 2 are exactly performed to receive accurate test result data.

The test result transmitter 23 transmits received test result data to the terminal 13 provided at the testing place 10, in order to allow the terminal 13 to instantly identify the test result.

In addition, the feedback module 24 receives the test result data from the test result receiver 22, and transmits the test result data to a learning module of the automatic analysis device 30 so that the automatic analysis device 30 updates an image analysis algorithm as shown in FIG. 3, in order to correct analysis accuracy of the automatic analysis device 30.

The image analysis management method for medical tests according to the embodiment of the present inventive concept as described above may be executed by an application (this may include a platform basically provided on a terminal or a program included in an operating system) that is basically installed on the terminal, or by an application (that is, a program) that a user downloads and installs by himself/herself from an application providing server such as an application store server, a web server associated with applications or corresponding services, etc. In this regard, the image analysis management method for medical tests according to the embodiment of the present inventive concept may be implemented as an application (that is, a program) basically installed on a terminal or installed by the user, and may be recorded on a computer-readable recording medium such as a terminal.

Such a program is recorded on a computer-readable recording medium and executed by a computer so that the aforementioned functions may be executed.

As described, in order to execute the image analysis management method for medical tests according to the embodiment of the present inventive concept, the above-mentioned programs may include codes which are coded with computer languages such as C, C++, JAVA, machine language, and the like which may be read by a processor (CPU) of the computer.

The code may include a function code associated with a function of defining the above-mentioned functions, and may also include an execution procedure related control code required to allow the processor of the computer to execute the above-mentioned functions as a predetermined procedure.

The code may further include a memory reference related code indicating at which location (address number) of the memory inside or outside the computer additional information or media required to allow the processor of the computer to execute the above-mentioned functions are referenced.

In addition, to allow the processor of the computer to execute the above-mentioned functions, when the processor needs to communicate with any other computers or servers, etc. at a remote location, the code may further include a communication-related code about how the processor of the computer communicates with any other computers or servers at a remote location or what information or media the processor of the computer transmits and receives at the time of the communication, by using the communication module (for example, wired and/or wireless communication module) of the computer.

In addition, a functional program for implementing the present inventive concept, a code and a code segment associated therewith, etc. may be easily inferred or changed by programmers in the art to which the present inventive concept pertains in consideration of a system environment of the computer which reads the recording medium and executes the program.

Examples of a computer readable recording medium recorded with programs as described above include a ROM, a RAM, a CD-ROM, a magnetic tape, a floppy disk, an optical media storage device, etc.

Also, a computer-readable recording medium recorded with programs as described above may be distributed to a computer system connected through a network, and thus stores and executes a computer-readable code in a distributed manner. In this case, at least any one computer among a plurality of distributed computers may execute a part of the above-mentioned functions and transmit the executed results to at least one of other distributed computers, and the computer receiving the result may also execute a part of the above-mentioned functions and provide the executed results to other distributed computers.

In particular, a computer-readable recording medium recorded with applications which are programs for executing the image analysis management method for medical tests according to the embodiment of the present inventive concept may be a storage medium (for example, a hard disk, etc.) included in an application store server or an application provider server such as a web server associated with applications or corresponding services, etc., or an application providing server itself.

A computer which may read a recording medium recorded with applications which are programs for executing the image analysis management method for medical tests according to the embodiment of the present inventive concept may include not only general PCs such as a typical desktop and a laptop, but also mobile terminals such as a smart phone, a tablet PC, personal digital assistants (PDAs), and mobile communication terminals, and is to be construed as all computable devices.

Also, when a computer which may read a recording medium recorded with applications which are programs for executing the image analysis management method for medical tests according to the embodiment of the present inventive concept is mobile terminals such as a smart phone, a tablet PC, a personal digital assistant (PDA), and a mobile communication terminal, the applications may be downloaded from an application providing server to a general PC and thus may also be installed on the mobile terminal by a synchronization program.

As described above, even though it is described that all constituent elements of the embodiment of the present inventive concept are combined as one or combined to be operated, the present inventive concept is not limited to the embodiment. That is, within the scope of purpose of the present inventive concept, at least one of constituent elements may be selectively combined to be operated. Also, all the constituent elements may be implemented as one independent piece of hardware, but a part or all of the constituent elements are selectively combined to be implemented as a computer program including a program module which performs a part or all functions and which is combined in one or a plurality of pieces of hardware. Codes and code segments which configure the computer program may be easily deduced by one of ordinary skill in the art. Such a computer program is stored in computer-readable media to be read and executed by the computer to implement the embodiment of the present inventive concept. A storage medium of the computer program may include a magnetic recording medium, an optical recording medium, etc.

Also, unless explicitly described to the contrary, the terms “comprise”, “configure”, and “have” should be understood to imply the inclusion of stated elements but not the exclusion of any other elements. Unless defined otherwise, all terms used herein, including technical terms and scientific terms, have the same meaning as commonly understood by a person of ordinary skill in the art to which various embodiments of the present inventive concept pertain. Terms defined in generally used dictionary shall be construed such that they have meanings matching those in the context of a related art, and shall not be construed as having ideal or excessively formal meanings unless they are clearly defined in the present inventive concept.

Although the embodiments have been described, those skilled in the art will readily appreciate that many modifications are possible without materially departing from the novel teachings and advantages. Therefore, the embodiments disclosed in the present inventive concept are not limited to the technical idea of the present inventive concept and are to explain these exemplary embodiments, and the scope of the technical idea of the present inventive concept is not limited by these exemplary embodiments. The protection range of the present inventive concept should be construed by the claims below, and all technical ideas within an equivalent range of thought should be construed as being included within the scope of the present inventive concept.

1. An image analysis management method for medical tests, the image analysis management method comprising:
receiving, by an image analysis management server for medical tests, an image of a sample photographed by a microscope, the sample being taken for a preset medical test, from a terminal provided at a testing place;
receiving test result data obtained by analyzing a received image through one of an analysis of an automatic analysis device and a manual analysis, from the automatic analysis device connected to the image analysis management server or a terminal of a manager performing the manual analysis; and
transmitting received test result data to the terminal provided at the testing place.

2. The image analysis management method of claim 1, wherein the receiving of the test result data comprises:
when test result data analyzed by the automatic analysis device is normally produced, receiving the test result data from the automatic analysis device; and
when test result data is not produced from the automatic analysis device that analyzes the received image, transmitting the received image to the terminal of the manager, and receiving test result data produced as a result of manual analysis of the received image from the terminal of the manager.

3. The image analysis management method of claim 2, further comprising, after the receiving of the test result data, transmitting the test result data received from the automatic analysis device or the terminal of the manager to a learning module of the automatic analysis device.

4. The image analysis management method of claim 3, wherein the automatic analysis device produces test result data by analyzing the received image through a preset image analysis algorithm, the image analysis management method further comprising, after the transmitting of the test result data to the learning module, updating the preset image analysis algorithm by using the test result data, by the automatic analysis device.

5. The image analysis management method of claim 1, wherein the preset medical test is one of a white blood cell classification and analysis test, a gynecologic cancer diagnosis test, and a chromosome analysis test.

6. An image analysis management server for medical tests, the image analysis management server comprising:
an image receiver configured to receive an image of a sample photographed by a microscope, the sample being taken for a preset medical test, from a terminal provided at a testing place;
a test result receiver configured to receive test result data that is obtained by analyzing a received image through one of an analysis by an automatic analysis device and a manual analysis, from the automatic analysis device or a terminal of a manager performing the manual analysis; and
a test result transmitter configured to transmit received test result data to the terminal provided at the testing place.

7. The image analysis management server of claim 6, wherein, when test result data analyzed by the automatic analysis device is normally produced, the test result receiver receives the test result data from the automatic analysis device, and when test result data is not produced from the automatic analysis device that analyzes the received image, the test result receiver transmits the received image to the terminal of the manager, and receives test result data produced as a result of manual analysis of the received image from the terminal of the manager.

8. The image analysis management server of claim 7, further comprising a feedback module configured to transmit the test result data received from the automatic analysis device or the terminal of the manager, to a learning module of the automatic analysis device.

9. The image analysis management server of claim 8, wherein the automatic analysis device produces test result data by analyzing the received image through a preset image analysis algorithm, and when the automatic analysis device receives the test result data from the feedback module, the automatic analysis device updates the preset image analysis algorithm by using the test result data.

10. A computer-readable recording medium having embodied thereon a program for executing an image analysis management method for medical tests, the image analysis management method comprising: receiving, by an image analysis management server for medical tests, an image of a sample photographed by a microscope, the sample being taken for a preset medical test, from a terminal provided at a testing place; receiving test result data obtained by analyzing a received image through one of an analysis of an automatic analysis device and a manual analysis, from the automatic analysis device or a terminal of a manager performing the manual analysis; and transmitting received test result data to the terminal provided at the testing place.
